(19) United States
(12) Patent Application Publication
(10) Pub. No.: US 2021/0128280 A1
(43) Pub. Date: May 6, 2021

(54) BLAST PROTOCOL

(71) Applicant: MILLENNIUM HEALTHCARE TECHNOLOGIES, INC., Cerritos, CA (US)

(72) Inventors: Robert H. Gregg, II, Huntington Beach, CA (US); Dawn M. Gregg, Huntington Beach, CA (US)

(21) Appl. No.: 17/087,393

(22) Filed: Nov. 2, 2020

Related U.S. Application Data
(60) Provisional application No. 62/929,103, filed on Nov. 1, 2019.

(51) Int. Cl. A61C 8/00; A61C 1/00

(52) U.S. Cl. CPC A61C 8/0087; A61C 1/0046; A61C 8/0089

(57) ABSTRACT

The BLAST Protocol is a tissue-sparing, tissue-integration, dental implant preparation, placement and maintenance protocol including use of a laser such as a free-running pulsed laser to irradiate the implant site before implant placement, irradiate the implant or implant fixture before implant placement, and irradiate the surgical site once the implant is placed.

### PRIORITY CLAIM AND INCORPORATION BY REFERENCE

This application claims the benefit of U.S. Prov. Pat. App. No. 62/929,103 filed Nov. 1, 2019 and entitled BLAST PROTOCOL.

This application incorporates by reference for all purposes the disclosure of U.S. patent application Ser. Nos. 14/940,126, 15/011,441, and 15/257,656 all of which are titled Laser-Assisted Periodontics and all of which include inventor Robert H. Gregg. This application incorporates by reference for all purposes the disclosure of U.S. Prov. Pat. App. No. 62/875,322 entitled Laser-Assisted Periodontics And Tooth Extraction which includes inventor Robert H. Gregg. This application incorporates by reference for all purposes the disclosure of U.S. Pat. Nos. 9,597,160 and 9,943,379.

### BACKGROUND OF THE INVENTION

### Field of the Invention

The present invention relates to dental methods. In particular, the present invention relates to steps performed in connection with dental surgery such as steps performed prior to, during, and after placement of a dental implant.

### Discussion of the Related Art

Tissue-sparing, tissue-integration, dental implant placement and maintenance procedures using lasers are in their infancy.

### SUMMARY OF THE INVENTION

The BLAST Protocol (“BLAST” or “Blast”) is a tissue-sparing, tissue-integration, dental implant preparation, dental implant placement and maintenance protocol. BLAST is a laser-based oral implant treatment protocol.

In various embodiments, Blast is designed to prepare the surgical site before, during, and after implant placement, enhance the biocompatible properties and increase the wettability of titanium implants, promote hemostasis, attenuate the inflammatory response, activate and upregulate growth factors, stimulate osteoblast viability and proliferation, improve bone-implant interface anchorage, shorten the implant healing period, and provide more predictable and more successful long-term implant placement outcomes.

The protocol may be used in conjunction with immediate implant placement after tooth extraction or avulsion, and during conventional implant procedures in healed edentulous sites. Portions of the protocol may also be used during periodic tissue maintenance recalls to reduce the occurrence of peri-implant mucositis and peri-implantitis.

Blast may involve methods and procedures including one or more of angiogenesis, bone disinfection, fibrin, fibroblast, growth factors, hemostasis, osseous regeneration, re-integration, re-osseointegration, selective photothermolysis, stem cells, and upregulation.

Biocompatibility improvement effects may include an increase in the hydrophilic characteristics (wettability) of titanium implants to increase the adhesivity and multidirectional spreading of osteoblasts along the surfaces, improved corrosion resistance of titanium implants, enhanced biocompatible properties of titanium implants and contributing to the downregulation of early inflammatory events, improved bone-implant interface anchorage, promotion of long-term hone bonding and interface strength, and creating titanium surfaces with greater cell adhesion abilities and improving bioactivity of titanium surfaces.

Anti-inflammatory efficacy may include blunting the lipopolysaccharide-induced inflammatory response, lowering immunological markers of inflammation (interleukin-1 beta (IL-1β) and tumor necrosis factor (TNF-α)) in gingival crevicular fluid, reducing major collagenase species (interleukin-1 beta (IL-1β) and matrix-metalloproteinase-8 (MMP-8)) in inflamed human periodontium, and attenuating inflammatory response by reducing lipopolysaccharide (LPS)-induced nitric oxide production and interleukin-8 production by endothelial cells.

Bactericidal capability may include removal of biofilm and cleaning contaminated implant surfaces, immediately suppressing red and orange complex periodontal pathogens below culture detection limits in most deep human periodontal pockets, ablating aerobic, anaerobic microbial species on implants without damaging the titanium surface.

Biostimulation effects may include stimulating osteoblast viability and proliferation, inducing expression of osteopontin, alkaline phosphatase, and Runt-related transcription factor 2 in osteoblasts, type I collagen in fibroblasts, and vinculin in endothelial cells, underlying molecular mechanisms demonstrative of a biostimulatory effect, stimulating bone regeneration by increasing osteoblast activity and accelerating mineral deposition, increasing new bone formation, and shortening the implant healing period by increasing bone interaction with hydroxyapatite-coated implants.

### BRIEF DESCRIPTION OF THE DRAWINGS

The present invention is described with reference to the accompanying figures. These figures, incorporated herein and forming part of the specification, illustrate the present invention and, together with the description, further serve to explain the principles of the invention and to enable a person skilled in the relevant art to make and use the invention.

FIGS. 1A-B show tables illustrative of some embodiments of the present invention.

FIGS. 2A-I show procedural steps illustrative of one or more embodiments of the present invention.

### DESCRIPTION OF THE PREFERRED EMBODIMENTS

The BLAST Protocol (“BLAST” or “Blast”) is a tissue-sparing, tissue-integration, dental implant preparation, dental implant placement and maintenance protocol. BLAST is a laser-based oral implant treatment protocol designed to prepare the surgical site before, during and after implant placement, enhance the biocompatible properties and increase the wettability of titanium implants, promote hemostasis, attenuate the inflammatory response, inhibit production of proinflammatory cytokines and prostaglandins, activate and upregulate growth factors, induce expression of genes related to osteogenesis, stimulate osteoblast viability and proliferation, improve bone-implant interface anchorage, shorten the implant healing period, and provide more predictable and more successful long-term implant placement outcomes.

The protocol may be used in conjunction with immediate implant placement after tooth extraction or avulsion, and during conventional implant procedures in healed edentulous sites. Portions of the protocol may also be used during periodic tissue maintenance recalls to reduce the occurrence of peri-implant mucositis and peri-implantitis.

### BLAST

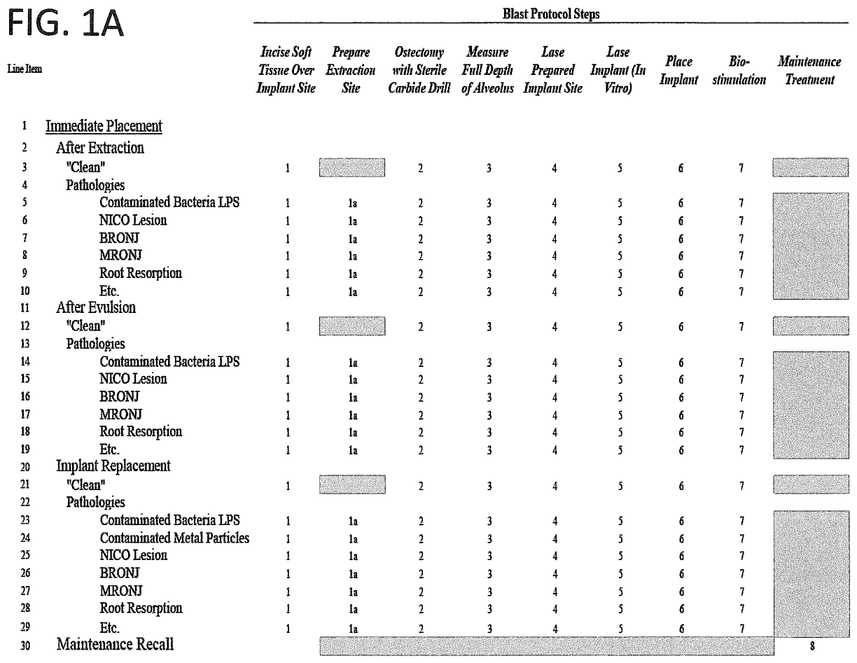

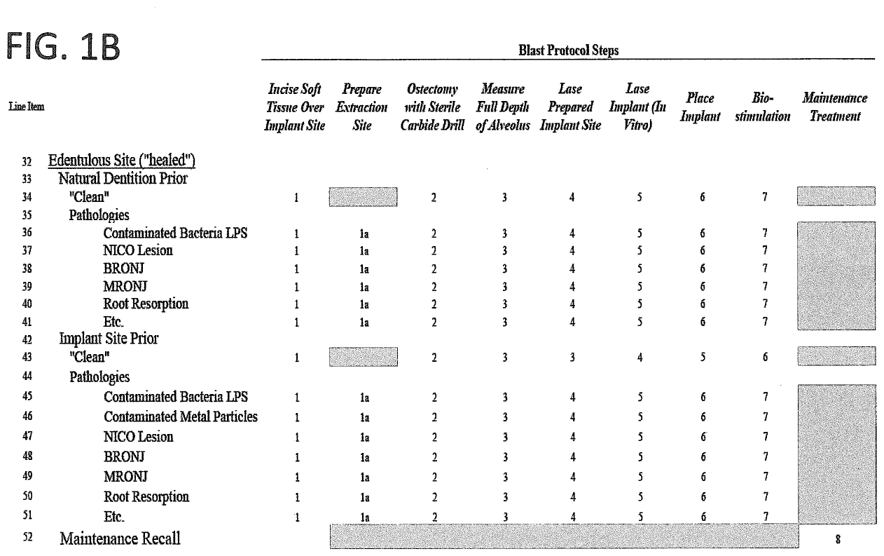

FIGS. 1A-B show tables that associate dental implant scenarios with related procedural steps of the Blast Protocol that may be used to accomplish each step. In general, as seen in FIG. 1A, a disturbed site may receive an implant immediately after or soon after an intentional or accidental removal of a tooth or implant from the site. Alternatively, as seen in FIG. 1B, an undisturbed site may receive an implant long after a tooth is removed and the site is healed over.

FIG. 1A tabulates placement of an implant after a tooth extraction (intentional), after a tooth evulsion (accidental), or after a previously placed implant is removed. Implant maintenance is also mentioned and discussed below.

Whether implant placement results from intentional, accidental, or replacement scenarios, procedural steps are aimed at cleaning the implant site and mitigating pathologies, including contamination with bacteria LPS (Lipopolysaccharide), NICO (Neuralgia-Inducing Cavitational Osteonecrosis) lesion, BRONJ (Bisphosphonate-Related Osteonecrosis of the Jaw), MRONJ (Medication-Related Osteonecrosis of the Jaw), root resorption, and the like. In the case of implant replacement, procedural steps may also include removal of contaminated metal particles.

Embodiments of the Blast Protocol include procedural steps for cleaning and mitigating these pathologies. For example, the Blast Protocol may include one or more of the following procedural steps in the order given or in a different order.

1. Incise soft tissue over implant site

2. Prepare extraction site

3. Perform osteotomy with sterile carbide drill or bur

4. Measure full depth of osteotomy site

5. Lase prepared implant site

6. Lase implant (In Vitro)

7. Place implant

8. Perform biostimulation

9. Perform maintenance treatment as needed

FIG. 1B tabulates placement of an implant at a healed site such as a site healed over following removal a tooth or removal of an implant. Implant maintenance is also mentioned and discussed below.

Whether placement of the new implant is replacement of a natural tooth of replacement of an implant, the procedural steps are aimed at cleaning the implant site and mitigating pathologies including contamination with bacteria LPS (Lipopolysaccharide), NICO (Neuralgia-Inducing Cavitational Osteonecrosis) lesion, BRONJ (Bisphosphonate-Related Osteonecrosis of the Jaw), MRONJ (Medication-Related Osteonecrosis of the Jaw), root resorption, and the like. In the case of implant replacement, procedural steps may also include removal of contaminated metal particles.

Embodiments of the Blast Protocol include procedural steps for cleaning and mitigating these pathologies. For example, the Blast Protocol may include one or more of the following procedural steps in the order given or in a different order.

1. Incise soft tissue over implant site

2. Prepare extraction site

3. Perform osteotomy with sterile carbide drill or bur

4. Measure full depth of osteotomy site

5. Lase prepared implant site

6. Lase implant (In Vitro)

7. Place implant

8. Perform biostimulation

9. Perform maintenance treatment as needed

### BLAST, Including Placement of a New Implant

BLAST procedures include multiple steps associated with placement and or maintenance of a dental implant. For example, BLAST may deal with placement of a dental implant following accidental loss of a tooth or with placement of a dental implant at an undisturbed site. The steps below describe a BLAST procedure for placing an implant.

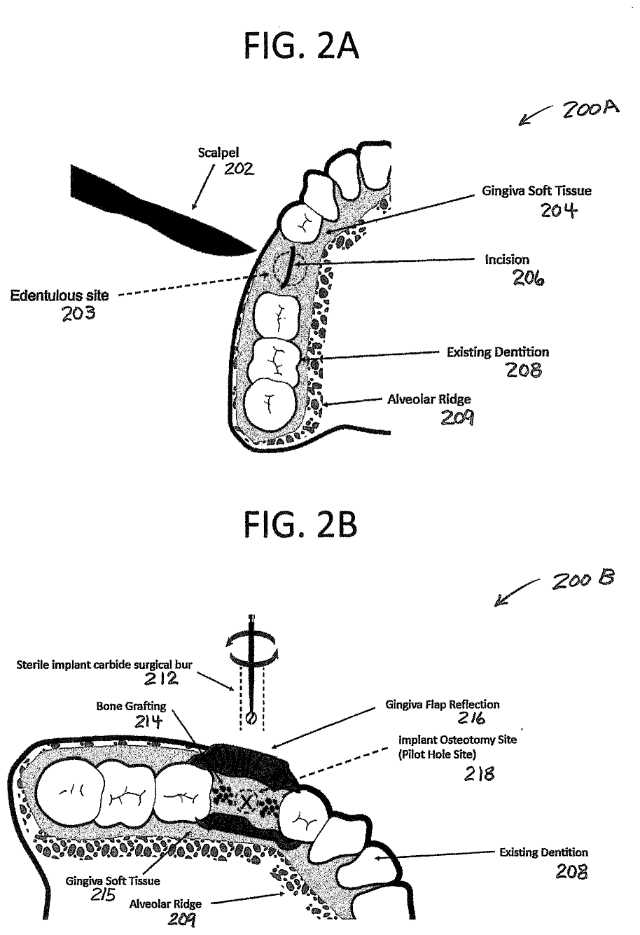

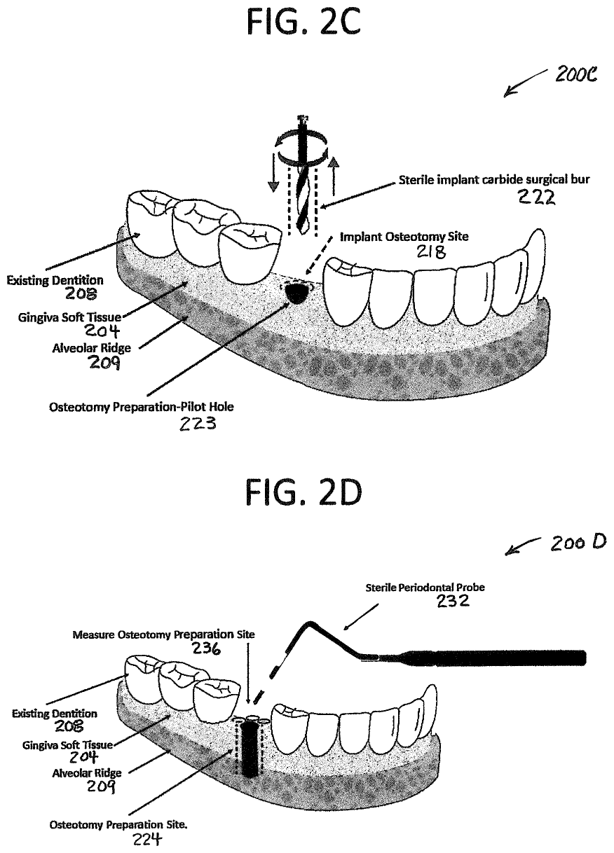

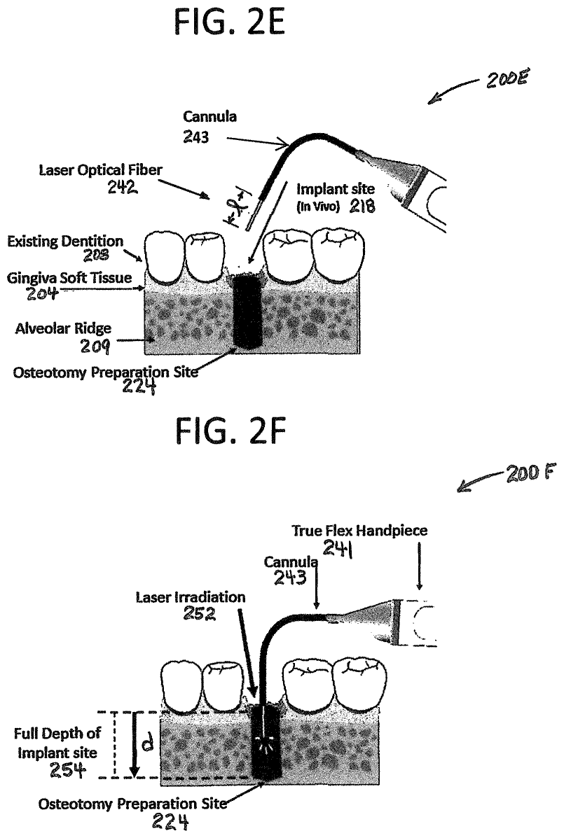

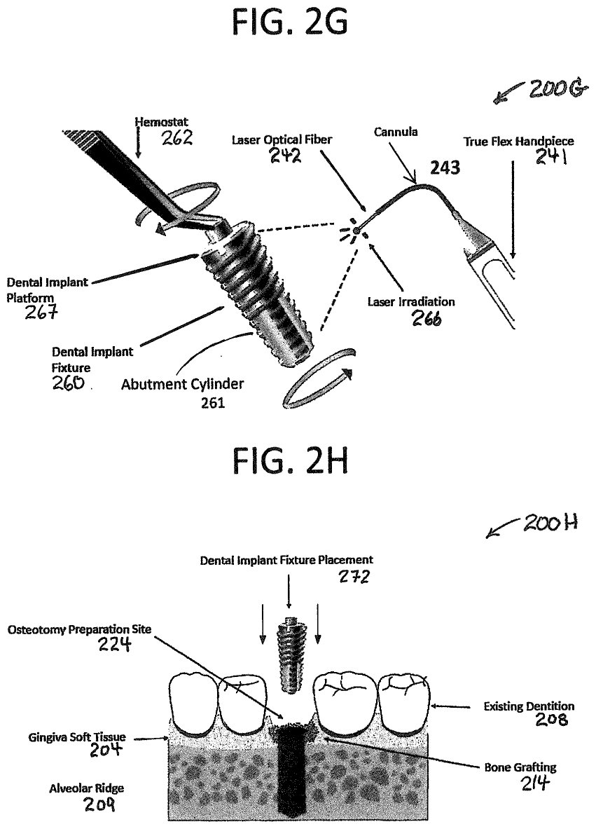

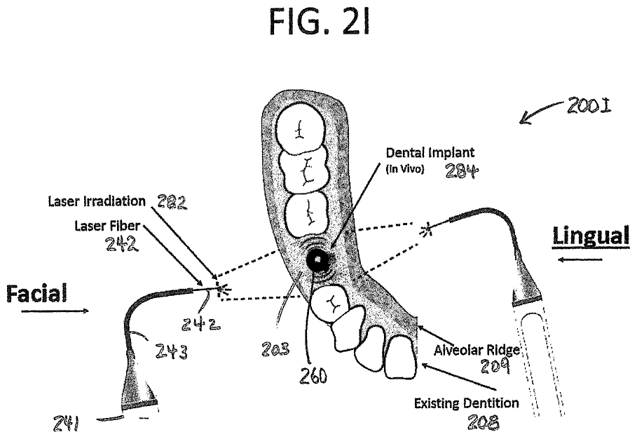

FIG. 2A shows a plan view of an undisturbed portion of a human patient's oral cavity 200A. Here, natural dentition (teeth) 208 are secured within an alveolar ridge 209 where osseous tissue (bone) is covered by intact soft tissue (mucosa) 204.

The empty (edentulate) site or space 205 between the teeth corresponds to a missing tooth, in this case a missing second premolar. Here, the edentulate site is readied to receive a dental implant, for example to replace a missing tooth. After suitable anesthesia has been administered, a sterile surgical scalpel 202 is used to create an incision 206 in the overlying mucosa 204 to expose the underlying bone.

FIG. 2B shows a plan view of a disturbed portion of a human patient's oral cavity 200B. Unlike FIG. 2A involving an undisturbed site, here tooth loss may be accidental with tissue surrounding the site of the missing second premolar upset in the process 211. Tooth loss may be the result of traumatic avulsion, tooth extraction, or both.

In a step which may be a first step (Step 1) involving either a healed site or an upset site, an implant or osteotomy site 218 surrounded by gingiva soft tissue 215 is surgically exposed by reflecting a gingiva soft tissue flap 216. Sterile implant drills and/or bone burs 212 are readied for use in creating an osteotomy site (e.g. for use in creating a socket or enlarged socket) in alveolar bone 209 that will receive the dental implant. Bone grafting materials 214 may be inserted into the site as the condition warrants to supplement the patient's existing alveolar bone 209.

FIG. 2C shows osteotomy site creation in the patient's jaw 200C. In a second step (Step 2), an ostectomy procedure is performed with sterile implant drills and/or burs 222 which may be of various dimensions to properly prepare for receiving an implant of a particular size or of various sizes. At the implant placement site 218, bone is removed or hollowed out 217 as osseous tissue is removed from alveolar bone 209. A pilot hole 223 may be created. and thereafter an osteotomy site that is a hollowed-out bone volume represented by the gray vertical cylinder 224 (see FIG. 2D) within the alveolar ridge.

FIG. 2D shows measurement of the osteotomy site created in the patient's jaw 200D. In a third step (Step 3), full depth “d” measurements of the osteotomy site 224 are made at specific points by means of a sterile periodontal probe 232. For example, 3 or more measurements may be made. For example, measurements may be evenly spaced or unevenly spaced, may be made at the deepest locations, or may be made at the shallowest locations. In some embodiments this procedure ensures the prepared site is unobstructed and/or of appropriate depth to enable the subsequent insertion of a particular dental implant such as a second premolar implant of a particular size.

FIG. 2E shows preparation for lasing the osteotomy site and surroundings 200E. Here, a laser includes a laser delivery system including, for example, a handpiece 241, a laser fiber extending from the handpiece 242, and a canula encasing a portion of the extending laser fiber. The fiber terminates in a free length “l” extending from the canula.

In a fourth step (Step 4), the laser fiber free length 242 is proximate the prepared implant site 218. The optical fiber is for transmitting laser energy to the implant site 218 as controlled by a clinician. The laser fiber free length 242 is adjusted using the measurements above to enable access and or energy transmission to a particular depth such as the maximum depth of the osteotomy site 224. In various embodiments, the laser's beam is not activated prior to its insertion into the osteotomy site.

FIG. 2F shows use of the laser to lase the osteotomy site and its surroundings 200F. In a fifth step (Step 5) the optical fiber free length 242 which may be flexible is inserted to the full depth 254 of the osteotomy site 224 and then the laser's beam is activated by the clinician 252. The laser's beam may be activated as the fiber is withdrawn from the site.

Heat generated by the pulsed laser beam initiates hemostasis. The process of inserting the free length 242 into the osteotomy site 224 and removal of the free length from the osteotomy site may be repeated until a desired amount of hemostasis or hemostasis condition is achieved. This process my simultaneously result in any one or more of activation of growth factors present in the blood, upregulation expression of genes related to osteogenesis to stimulate osteoblast viability and proliferation, and inhibition of production of proinflammatory cytokines and prostaglandins to shorten the implant healing period.

FIG. 2G shows in vitro laser irradiation prior to implant insertion 200G. In a sixth step (Step 6) a sterile titanium dental implant 260 is irradiated 266 prior to insertion into the osteotomy site 224. The implant may be held with forceps 262 near the dental implant platform 267 and may be used to turn 264 the implant. Notably, in some embodiments the dental implant may be made from one or more materials such as metal(s) which may include titanium or not.

In some embodiments, the entire surface of the implant below the abutment cylinder 261 is irradiated by the pulsed laser beam via an attached optical fiber. The optical fiber may be held out-of-contact with the implant surface. This procedure enhances the hydrophilic (wettability) properties of the implant to increase the adhesivity and multidirectional spreading of osteoblasts along the implant surfaces, thereby improving bone-implant interface anchorage.

FIG. 2H shows the implant ready for placement 200H. In a seventh step (Step 7) the irradiated implant 260 is located proximate 272 the osteotomy site 224 in the alveolar ridge 209. Bone grafting materials 214 may be inserted into the site as the condition warrants to supplement the patient's existing alveolar bone 209.

FIG. 2I shows the implant inserted in the osteotomy site and biostimulation 200I. In an eight step (Step 8) the irradiated implant 260 is inserted 284 to the appropriate depth within the osteotomy site 224.

After the implant 260 is inserted in the osteotomy site 224, the clinician activates the laser's beam as the optical fiber free end 242 is aimed toward, but remains out-of-contact with, the implant 260 and/or surroundings 203 from both facial and lingual aspects. Here, the laser's emission/photonic energy penetrates into the adjacent tissues. In various embodiments, results may include one or more of laser-induced biostimulation that stimulates bone regeneration by increasing osteoblast activity and accelerating mineral deposition, shortening the healing period of the soft and osseous tissues in implant site, thereby providing a more predictable and more successful long-term implant placement outcome.

### BLAST, Including Peri-Implant Infection of an Existing Implant

Peri-implant infection and inflammation are caused by certain types of bacteria in plaque and calculus (concrements). These bacteria create toxins which irritate the gums, cause deep pockets, and result in a breakdown of the attachment of bone to implants. Over time, these toxins can destroy gum tissues, allowing the infection to progress, and can result in bone loss.

Accordingly, there is a need for a minimally invasive surgical method for the removal of a deep pocket, elimination of disease, reattachment of the gingiva to the implant surface and re-osseointegration of the implant.

Therefore, according to one example embodiment described herein, dental disorders associated with a dental implant are treated. An average power for a laser is selected by a user interface on a display, along with a set of permissible laser parameters provided in response to the selected average power. A gingival trough or flap is created around the implant with the laser. Infected tissue is selectively ablated or denatured via selective photothermolysis, and a pocket is lased around the affected implant. Corrosion products are removed, and steps are performed to create and maintain angiogenesis. Marginal tissues are compressed against the implant and occlusal interferences are removed.

By virtue of this arrangement, it is ordinarily possible to treat mucositis and peri-implantitis while reducing peri-implant pocket defects, by establishing a new connective tissue attachment to the implant at, or near, the coronal level, and re-osseointegration of the implant.

According to one aspect, a selection of an average power for a laser is received via a user interface on a display device, and a set of permissible laser parameters is provided to the display device and laser head in response to the selected average power. The laser head is controlled in accordance with the laser parameters to create a gingival trough or flap around an implant, ablate or denature infected tissue via selective photothermolysis, and lase a pocket around the infected tissue.

According to still another example aspect, ablating or denaturing the infected tissue includes ablating or denaturing inflamed, infected, erythematous, edematous, hyperplastic, ulcerated, degenerated, bleeding, suppurative, or sloughing periodontal or peri-implant soft tissue, including sulcular epithelium, junctional epithelium, and keratinized tissue, via selective photothermolysis.

The laser device is a laser for performing laser therapy including laser dentistry (e.g., ablation of bacteria in gum tissue, reducing contamination on dental implants). Exemplary lasers may be integrated in a handpiece or a handpiece may extend from a lasing device via a fiber optic umbilical. For example, the laser might correspond to a “PerioLase®MVP-7™”, manufactured by Millennium Dental Technologies, Inc. In that regard, the PerioLase® MVP-7™ is a 6-Watt FR (Free Running) Nd:YAG (Neodymium:Yttrium-Aluminum-Garnet) laser with features necessary to perform soft tissue procedures, and includes operator-selectable pulse durations from, e.g., 100 to 650 microseconds (μsec) to allow optimum ablation and hemostasis.

Peri-implant infection and inflammation and peri-implant diseases are caused by certain types of bacteria in plaque and calculus (concrements). These bacteria create toxins which irritate the gums and result in a breakdown of the attachment of the bone to the implants. Over time, these toxins can destroy gum tissues, allowing the infection to progress, and can result in bone loss. There are many forms of peri-implant diseases, the most common types being peri-implant mucositis and peri-implantitis. Peri-implant mucositis are the earliest stage and affect only the gum tissue. At this stage, the disease is still reversible.

If not treated, however, peri-implant mucositis may lead to a more severe condition called peri-implantitis. The gums, bone and other structures that support the implants become damaged. Implants can become loose and may have to be removed. At this stage, the disease may require more complex treatment to prevent implant loss. With healthy gingiva (gum tissue), the implants are firmly anchored in bone. Peri-implant mucositis develops as toxins in plaque irritate the gums, making them red, tender, swollen, and likely to bleed easily. Peri-implantitis occurs when toxins destroy the tissues and bone. Gums become detached from the implants, forming pockets that fill with more plaque. Advanced peri-implantitis is present when the implants lose the supporting bone. Unless treated, the affected implant frequently becomes loose and may fall out.

Conventionally, the first step in the treatment of peri-implantitis is usually a thorough cleaning which may include scaling to remove plaque and calculus deposits beneath the gum line. Surgery may be required when deeper pockets, usually over 4 to 6 mm, are found. It is difficult for the dentist or hygienist to thoroughly remove plaque and calculus from deep pockets. Patients can seldom keep them clean and free of plaque. Allowing pockets to remain may invite infection and bone destruction.

When pockets are deep and bone has been destroyed, flap surgery may be necessary to provide access to the surfaces of the implants in order to thoroughly remove calculus, plaque and any diseased tissue, and to recontour the bone to a more favorable architecture. In this technique, the gum is lifted away and is then sutured back into place or into a new position for ease of cleaning.

Conventionally, surgical debridement of the implant surface and the removal of granulation and granulomatous tissue are performed following the resection of the soft tissue flap. Aesthetic modifications of this approach have been reported under the titles such as open flap curettage, reverse bevel flap surgery, Widman flap surgery and modifications of Widman flap surgery, apically positioned flap osseous surgery, and guided tissue regeneration.

Nevertheless, conventional methods lack an appropriate minimally invasive surgical method for the reduction of the deep pocket, elimination of disease, reattachment of the gingiva to the implant surface and re-osseointegration of the implant. Exemplary embodiments for addressing these issues are described below.

### BLAST, Including Laser-Based Implant Maintenance Treatment of Existing Peri-Implant Disease

Peri-implant mucositis and peri-implantitis may include reducing early, shallow and deep bony pockets to remove of diseased tissue, peri-implant pathogens, pathologic proteins, calculus and other concrements on the implant surface, and corrosive by-products of metal implant degradation. This provides for regrowth, regeneration, and re-integration of new bone to the implant fixture. Notwithstanding the above, it should be noted that not all implants are made of titanium (e.g., ceramic), and the process may apply to such other types of implants.

The process may include creating a gingival trough or flap around the implant with a contact laser fiber (after first removing the prosthetic crown if possible), and selectively ablating or denaturing the infected and inflamed pocket epithelium via selective photothermolysis. The process may further includes vaporizing or denaturing the inner marginal gum tissues and pocket epithelium and granulomatous tissue fully around the targeted implant to the accessible depth of the defect without breaking through the soft tissue attachment apparatus above the depth of the bony defect, ultrasonic debridement of the implant surfaces, transitioning to the full depth of the bony defect via blunt dissection through any soft tissue attachment and perforating into the bony defect, modifying the bone through osteoplasty and/or ostectomy below the level of the mucoperiosteum as needed, creating angiogenesis, lasing the pocket to disinfect and decontaminate the soft and hard tissues and implant, assisting in hemostasis, cauterizing free nerve endings, sealing lymphatics, preparing the coronal soft tissue for approximation against the implant, and compressing the soft marginal tissues against the implant until blood flow has ceased, adhesion is achieved, and a stabilized fibrin clot has formed. In one example, elimination of traumatic occlusal forces is typically achieved by removal of the implant-retained restoration or occlusal adjustment if removal is not an option.

By virtue of this arrangement, it is ordinarily possible to treat peri-implant mucositis and peri-implantitis peri-implant pocket defects by establishing a new connective tissue attachment to the implant at, or near, the coronal level. Moreover, the inflamed pocket epithelium is selectively separated via photothermolysis, ordinarily without substantially removing any connective tissue.

In that regard, a topically placed anesthetic is used to anesthetize the area. In one example, the dentist may begin with 4% prilocaine plain, using a 30-gauge needle. This anesthetic is perceived by the patient as painless, due to its unique ability to anesthetize soft tissue without stinging. The anesthetic is injected very slowly into the area, allowing several minutes for the prilocaine plain to take effect. The dentist may then continue using a 30-gauge needle and follow this procedure with a suitable longer-acting anesthetic. However, an exception would be made if health reasons caused the anesthetic to be contraindicated. The area of concern usually involves one to three implant fixtures and could be combined in conjunction with the LANAP® Protocol treatment of two quadrants, or alternatively, one arch, either upper or lower. Anesthesia is routinely used in every procedure, in order to: aid in bone-sounding (discussed below) for accurate measurement of the full depth of the diseased pocket and bony defects; allow aggressive debridement of soft and hard tissues around the surfaces of the implant; allow the patient to be as comfortable as possible during the treatment, thereby minimizing the patient's endogenous adrenaline production, and in turn achieve the optimal therapy results; maximize the doctor's ability to concentrate on the procedure; and optimize the use of ultrasonic probes at frequencies between one hertz and fifty thousand hertz.

As another preliminary step, bone sounding and pocket depth measurement can be performed using a periodontal probe, recording the depths of all bony defects in the soft tissue around the implant, from an upper gingival margin to the extent of the accessible bony defect. In one example, pocket depths can be recorded with a periodontal probe with six areas recorded around each implant. This will allow a determination of the full depth of the diseased pocket. The dentist uses the sum total of all 6 probe depths/bone soundings and multiplies that number by 4 to compute a “light dose” of 4/Joules per millimeter pocket depth. (For example: 6 probe depths of 10 mm each 60 mm total×4=240 Joules of total light dose.) The summation number of the probe depth represents the TOTAL Joules to be delivered. The total light dose is applied ⅔rds during the 1st Step of laser application in LAPIP™ Ablation, while the remaining ⅓ of the energy is delivered during the 2nd laser application in the LAPIP™ Hemostasis setting. (In the example above, 160 Joules are delivered during the LANAP® Ablation Step, and Joules are delivered during the LAPIP™ Hemostasis Step). Thus, a light dose computation is made in conjunction with the surgical treatment.

Ablation is performed. In this regard, ablation of the free gingival margin with the laser energy removes pathogens and pathologic proteins within the tissue of the free margin, which otherwise would not be removable, whereas lasing the implant surface is used to, e.g., remove only granulomatous tissue, intentionally leaving the disinfected granulation tissue in place, and to disinfect, assist in hemostasis, cauterize free nerve endings, and seal lymphatics of the pocket tissue surface.

Cleaning is performed with, e.g., an ultrasonic handpiece, along with further cleaning by a laser delivery system. In particular, the implant surface is cleaned of all foreign matter, to the full depth of the pocket on all sides of the implant from crestal margin to bony base. For example, the dentist may use an ultrasonic handpiece to ultrasonically scale all implant surfaces to the depth of the pocket, with the intent to remove all foreign structures and substances from the implant surface (including calculus and cement), thereby allowing adhesion of the lased soft tissue to the clean implant surface. Bone modification, as appropriate with osteotomy and/or ostectomy, may be undertaken. Then, using a laser delivery system, between one and six Watts of laser fiber output power and a frequency between one hertz and one hundred hertz may be used in the deep periodontal pockets for optimal bacterial destruction without causing bacterial injection into the periodontal tissues. This will minimize the occurrence of soft tissue cellulitis.

Lasing is performed with a laser delivery system, to remove only granulomatous tissue, intentionally leaving the disinfected granulation tissue in place, and to disinfect, assist in hemostasis, cauterize free nerve endings, and seal lymphatics of the pocket tissue surface, and to prepare the pocket tissue surface for adhesion. The laser delivery system may also be used to stop blood flow as needed.

In one specific example, although the disclosure is not hereby limited, a laser delivery system might comprise a FiberFlex™ 360-micron diameter quartz optical fiber feed through a handpiece such as an anodized aluminum True-Flex® handpiece and annealed stainless-steel cannula. The dentist activates the laser to intentionally irradiate the bone at the base of the bony defect in the 6 separate pocket depth measurement locations to initiate hemostasis from the medullary bone, stimulate and upregulate the release of growth factors (e.g., IGF-I and IGF-II, TGF-beta 1, TGF-beta 2, BMP-2), stimulate and upregulate fibroblasts and stem cells, warm the blood in the pocket to thermolytically cleave fibrinogen thereby converting the blood into fibrin (thrombin catalyzes the conversion of fibrinogen to fibrin), the body's first connective tissue, create a stable fibrin clot, and to create angiogenesis (new vascularization); to remove and/or denature any remaining, residual granulomatous tissue, and inflamed, infected and diseased epithelial lining, intentionally leaving granulation tissue in place (stems cells, capillaries, fibroblasts), but disinfected; and to, e.g., cauterize free nerve endings and seal lymphatics of the pocket tissue surface, and to prepare the pocket tissue surface for adhesion.

The procedure can be categorized as a Surgical Flap Procedure and “Laser-Assisted Regeneration”, with limited or complete occlusal adjustment. In some examples, a time of 20 minutes is reasonable to treat a single implant fixture if crown removal is not involved. As suggested above, treatment can be followed by a coronal polishing/prophylaxis and an occlusal equilibration follow-up and a postoperative check of the area treated.

According to still another example aspect, ablating or denaturing the infected tissue comprises ablating or denaturing inflamed, infected, erythematous, edematous, hyperplastic, ulcerated, degenerated, bleeding, suppurative, or sloughing periodontal or peri-implant soft tissue, including sulcular epithelium, junctional epithelium, and keratinized tissue, via selective photothermolysis.

Further example aspects of one embodiment of an example procedure are now described. In one example, the area of concern, usually two quadrants, is anesthetized. The procedure is applied independently to each implant involved. Pocket depth is measured and recorded with a perio probe to determine the full depth of the diseased pocket. A contact laser fiber is oriented along the long axis of the implant and is used to create a gingival trough or flap by ablating the free gingival margin and the internal epithelial lining of the pocket, thereby exposing the implant surface. Appropriately cleaved contact laser fibers provide precise control of the laser energy, the physical placement of the laser energy, and the determination of the desired physical orientation of the laser to the tissue to be removed. A gingival trough or flap is used to expose the implant surface for visualization. Excision of the free gingival margin removes pathogens and pathologic proteins within the tissue of the free margin which are otherwise unremovable and provides hemostasis for better visualization. This step also defines the tissue margins preceding mechanical instrumentation and preserves the integrity of the mucosa by releasing tissue tension. It also dissects-out the separation between the free gingival margin and the fibrous collagen matrix which holds the gingiva in position. This aids in the maintenance of the crest of the gingival margin. With the use of the “hot-tip” effect, further excision of the inner pocket epithelium around the entire implant is completed, to the depth of the probe readings. Ordinarily, no attempt is made to break through the mucogingival junction with the optical fiber. The “hot-tip” effect (accumulated tissue proteins heated via conductivity secondary to the passage of laser energy through the fiber) provides the selective removal of sulcular and pocket epithelium and granulomatous tissue without substantially removing any connective fibrous tissue and does so circumferentially and radially. As necessary, the excised tissue that accumulates on the tip of the laser fiber is removed. Ultrasonic scaling of all implant surfaces to the depth of pocket is completed. The intent is to remove all foreign structures and substances from the pocket to allow adhesion of the soft tissue to the clean implant surface. Lasing of the pocket to remove remaining granulomatous tissue, disinfect tissue, assist in hemostasis, cauterize free nerve endings, seal lymphatics, prepare tissue for soft and fibrin clot adhesion to implant surface is accomplished.

Elimination of occlusal interferences is completed using e.g., a high-speed handpiece as described herein. For best results this step is helpful, since it allows the tissue to heal and the bone to regenerate. The laser modifies the tissue to allow new attachment to take place but if the trauma of malocclusion continues the tissue cannot withstand and begins to break down immediately. All treatment sites are irrigated to the deepest depth of the periodontal pockets with a bactericidal solution of a high tissue substantivity (e.g., chlorhexidine gluconate 0.12%). The irrigation aids the laser in the reduction of bacteria in the pocket and in removing debris. Approximation of the wound edges is completed. Lasing is further accomplished to control blood flow as needed. Healing of the wound edges is by secondary intention. The tissue is compressed with finger pressure for 1 to 3 minutes against the implant from both a facial and lingual direction in order to permit only a thin clot to form between the tissue and the implant.

Post-procedural steps include prescribing medications for home use and reviewing postoperative care with the patient. An occlusal splint may be used to provide anterior guidance, e.g., a “QuickSplint®”, or anterior “jig”. A thorough occlusal adjustment follow-up examination is required. This treatment should continue periodically until bone development is complete. Pocket-depth measurements are to be avoided for 12 months.

In another example embodiment, a laser-assisted peri-implant mucositis and peri-implantitis bone regeneration and re-osseointegration procedure uses a free-running pulsed neodymium:yttrium-aluminum-garnet laser device with a 1,064-nanometer wavelength and duty cycles between 0.2 and 1.3 percent (100 and 650 microseconds at 20 hertz), Average Power of 3.0 Watts, 150 millijoules, Peak Power of 1500 Watts/pulse, Energy Density of 147 J/cm², Power Density of 2947 Watts/cm² to an Average Power of 3.6 Watts, 180 millijoules, Peak Power of 1800 Watts/pulse, Energy Density of 177 J/cm², Power Density of 3537 Watts/cm² using preferably the free-running pulsed Nd:YAG PerioLase® MVP-7™ and includes steps of anesthetizing mucogingival tissues corresponding to a targeted implant of a patient, the implant having an implant surface, bone sounding using a periodontal probe and recording the depths of all bony defects in the soft tissue at 6 sites around the implant and to bone, from an upper gingival margin to the extent of the accessible bony defect, recording the sum total of all 6 probe depths/bone soundings and multiplying by a predesignated constant which in this example is 4 (representing a “light dose” of 4 Joules per millimeter pocket depth. Example: 6 probe depths of 10 mm each=60 mm total 4=240 Joules of total light dose).

The total light dose is applied such that the majority of the total light dose is applied during the 1st step of laser application in LAPIP™ Ablation, while the remaining portion of the total light dose is delivered during the 2nd laser application in the LAPIP™ Hemostasis setting. In this example, the total light dose is applied ⅔rds during the 1st step of laser application in LAPIP™ Ablation, while the remaining ⅓rd of the energy is delivered during the 2nd laser application in the LAPIP™ Hemostasis setting. In this example, 160 Joules are delivered during the LAPIP™ Ablation Step, and 80 Joules are delivered during the LAPIP™ Hemostasis Step. The procedure further uses average powers of 3.0 to 3.6 Watts, 20 hertz repetition rate, and 100-microsecond pulse duration with a 0.2 percent duty cycle. Average Power of 3.0 Watts, 150 millijoules, Peak Power of 1500 Watts/pulse, Energy Density of 147 J/cm², Power Density of 2947 Watts/cm² to an Average Power of 3.6 Watts, 180 millijoules, Peak Power of 1800 Watts/pulse, Energy Density of 177 J/cm², Power Density of 3537 Watts/cm².

The example further uses a FiberFlex™ 300-, 320-, 360-, 400-micron (preferably a 360-micron) diameter quartz optical fiber fed through an anodized aluminum TrueFlex® handpiece and annealed stainless steel cannula, ablating, denaturing and vaporizing granulomatous tissues, inflamed, infected, ulcerated epithelial lining of the pocket, photothermally altering, disrupting, denaturing, dehydrating, and destroying hard calcified calculus and concrements on the implant surface, to the soft tissue extent of the pocket on all sides of the implant to prepare a new and coronal crestal surface for connective tissue adhesion and osseointegration, and includes lasing the implant surface to destroy lipopolysaccharides (LPS) of gram-negative bacteria, using a laser and/or preferentially LANAP® piezoelectric ultrasonic device with water lavage and 20,000 to 30,000 hertz, and three specific tips-the “P” tip, the “Ball” tip, and the “PS” tip operating at 8 to 10 Watts, cleaning the implant surface of all foreign matter, calculus, and cement to the full depth of the pocket on all sides of the implant from crestal margin to bony defect base, decorticating the crestal and marginal ridge bone to perform an osteotomy and/or ostectomy and to initiate angiogenesis, irrigating the pocket with a bactericidal solution, preferably chlorhexidine 0.12%, and using the laser with average powers of 3.0 to 4.0 Watts, 20 hertz repetition rate, and 150- to 650-microsecond pulse duration, preferably with Duty Cycles between 0.3 percent and 1.3 percent. At 150-microsecond pulse duration: Average Power of 3.0 Watts, 150 millijoules, Peak Power of 1000 Watts/pulse, Energy Density of 147 J/cm², Power Density of 2947 Watts/cm² and Duty Cycle of 0.3 Percent; to an Average Power of 4.0 Watts, 180 millijoules, Peak Power of 1333 Watts/pulse, Energy Density of 196 J/cm², Power Density of 3930 Watts/cm² and Duty Cycle of 0.3 percent; to 650-microsecond pulse duration: Average Power of 3.0 Watts, 150 millijoules, Peak Power of 231 Watts/pulse, Energy Density of 147 J/cm², Power Density of 2947 Watts/cm² and Duty Cycle of 1.3 Percent; to an Average Power of 4.0 Watts, 180 millijoules, Peak Power of 307 Watts/pulse, Energy Density of 196 J/cm², Power Density of 3930 Watts/cm² and Duty Cycle of 1.3 percent.

In one aspect, the procedure further includes using a FiberFlex™ 300-, 320-, 360-, 400-micron (preferably a 360-micron) diameter quartz optical fiber fed through an anodized aluminum TrueFlex® handpiece and annealed stainless steel cannula; lasing to intentionally irradiate the bone at the base of the bony defect in the 6 separate pocket depth measurement locations to initiate hemostasis from the medullary bone; stimulate and upregulate the release of growth factors (e.g., IGF-1 and IGF-II, TGF-beta 1, TGF-beta 2, BMP-2); stimulate and upregulate fibroblasts and stem cells; warm the blood in the pocket to thermolytically cleave fibrinogen thereby converting the blood into fibrin (thrombin catalyzes the conversion of fibrinogen to fibrin), create a stable fibrin clot, and create angiogenesis; remove and/or denature any remaining, residual granulomatous tissue and inflamed, infected and diseased epithelial lining, intentionally leaving granulation tissue in place (stem cells, capillaries, fibroblasts), but disinfected; disinfect, assist in hemostasis, cauterize free nerve endings, and seal lymphatics of the pocket tissue surface; prepare the pocket tissue surface for adhesion; lasing the pocket tissue surface to adapt the pocket tissue surface for tissue adhesion; Average Power of 3.0 Watts, 150 millijoules, Peak Power of 1000 Watts/pulse, Energy Density of 147 J/cm², Power Density of 2947 Watts/cm² to an Average Power of 4.0 Watts, 180 millijoules, Peak Power of 1333 Watts/pulse, Energy Density of 196 J/cm², Power Density of 3930 Watts/cm², approximating the pocket tissue surface with the implant surface; maintaining the pocket tissue surface in contact with the implant surface to advance adhesion; and eliminating occlusal interferences.

In one aspect, the depth measuring is completed with a periodontal probe taking at least six spaced-apart measurements around the implant. In another aspect, the ablating, vaporizing, and lasing are completed with a laser fiber oriented parallel to the surface of the implant.

In still another aspect, the procedure includes a step of providing a free-running pulsed Nd:YAG, 1,064-nanometer wavelength laser, preferably the PerioLase® MVP-7™, wherein the ablating, denaturing and vaporizing is completed with not more than 6.00 Watts of average output power from the laser, as measured at the distal end of the fiber, and with a lasing frequency of not more than 100 Hz. Average Power of 3.0 Watts, 150 millijoules, Peak Power of 1500 Watts/pulse, Energy Density of 147 J/cm², Power Density of 2947 Watts/cm² to an Average Power of 3.6 Watts, 180 millijoules, Peak Power of 1800 Watts/pulse, Energy Density of 177 J/cm², Power Density of 3537 Watts/cm².

In yet another aspect, the laser fiber is of a diameter between approximately 200 and 600 microns. In still another aspect, the method includes firm pressure to hold the pocket tissue surface in contact with the implant surface for 1 to 3 minutes allowing a thin clot to form between the pocket tissue surface and the implant surface.

In some embodiments: A laser-assisted peri-implant mucositis and peri-implantitis bone regeneration and re-osseointegration procedure using a free-running (FR) pulsed neodymium:yttrium-aluminum-garnet (Nd:YAG) laser device with a 1,064-nanometer wavelength and operating over a preferred parameter range which includes duty cycles between 0.2 and 1.3 percent (preferably at 100 to 650 microseconds at 100 hertz or less, preferably 20 hertz); average power of less than 10 Watts, preferably between 3.0 Watts and 3.6 Watts, 150 millijoules and 180 millijoules; peak power of between 231 Watts/pulse and 1800 Watts/pulse; energy densities of between 147 J/cm² and 177 J/cm²; and power densities of between 2947 Watts/cm² and 3537 Watts/cm², the procedure comprising: a) anesthetizing mucogingival tissues corresponding to a targeted implant of a patient, the implant having an implant surface; b) bone-sounding in a pocket around the targeted implant using a periodontal probe, and recording the depths of all bony defects in the soft tissue at 6 sites around the implant and to bone, from an upper gingival margin to the extent of the accessible bony defect; c) recording the sum total of all 6 probe depths/bone soundings and multiplying by 4 so as to obtain a total light dose estimation in units of Joules to be delivered; d) ablating, denaturing and vaporizing the inner diseased, inflamed, infected, ulcerated epithelial lining of the pocket, implant corrosion products and granulomatous tissues so as to photothermally alter, disrupt, denature, dehydrate and destroy hard calcified calculus and concrements on the implant surface, to the soft tissue extent of the pocket on all sides of the implant to prepare a new and coronal crestal connective tissue and osseointegrative surface, wherein said step of ablating, denaturing and vaporizing comprises application of ⅔ of the total light dose estimation using a quartz optical fiber having a small diameter of less than or equal to 400 microns and fed through a handpiece and an annealed stainless steel cannula, while operating the FR Nd:YAG laser device within the preferred parameter range; e) lasing the implant surface to destroy and denature lipopolysaccharides (LPS) of gram-negative bacteria f) cleaning the implant surface of all foreign matter, calculus, cement to the full depth of the pocket on all sides of the implant from crestal margin to bony defect base, wherein said step of cleaning comprises application of a laser and/or preferentially piezoelectric ultrasonic device with water lavage and 20,000 to 30,000 hertz and use of appropriate tips operating at 8 to 10 Watts; g) decorticating the crestal and marginal ridge bone to perform an osteotomy and/or ostectomy, and to initiate angiogenesis; h) irrigating the pocket with a bactericidal solution, preferably chlorhexidine 0.12%; i) lasing to intentionally irradiate the bone at the base of the bony defect in the 6 separate pocket depth measurement locations to initiate hemostasis from the medullary bone, stimulate and upregulate the release of growth factors such as IGF-I and IGF-II, TGF-beta 1,TGF-beta 2, BMP-2, and stimulate and upregulate fibroblasts and stem cells, warm the blood in the pocket to thermolytically cleave fibrinogen thereby converting the blood into fibrin (thrombin catalyzes the conversion of fibrinogen to fibrin), create a stable fibrin clot, and create angiogenesis; remove and/or denature any remaining residual granulomatous tissue and inflamed, infected and diseased epithelial lining, intentionally leaving granulation tissue in place inclusive of stem cells, capillaries, fibroblasts, but disinfected, and disinfect, assist in hemostasis, cauterize free nerve endings, and seal lymphatics of the pocket tissue surface, and prepare the pocket tissue surface for adhesion; j) lasing the pocket tissue surface to adapt the pocket tissue surface for tissue adhesion; wherein said steps (i) and (j) of lasing comprise application of the remaining ⅓ of the total light dose estimation using small diameter quartz optical fiber fed through a handpiece and annealed stainless steel cannula, while operating the FR Nd:YAG laser device within the preferred parameter range with the differentiated exception of average power of 3.0 to 4.0 (not 3.6) Watts and a duty cycle of 150 (not 100) to 650 microseconds at 20 hertz; k) approximating the pocket tissue surface with the implant surface; 1) maintaining the pocket tissue surface in contact with the implant surface to advance adhesion; and m) eliminating occlusal interferences.

Pocket depths may be measured using a periodontal probe around the implant, and wherein a predesignated constant is used such that the total light dose estimation, in units of Joules to be delivered, is obtained by multiplying the sum total millimeters of probe depths/bone soundings by a predesignated constant.

The method above may include lasing to intentionally irradiate the bone at the base of the bony defect, said step of lasing further stimulates and upregulates the release of growth factors, and stimulates and upregulates fibroblasts and stem cells.

The method above may be accomplished using a blue light device with wavelength emission in the range of 400 to 520 nm (e.g., 405, 420, 425, 470 nm), such as a diode laser, Ti:sapphire laser, argon ion laser, light-emitting diode, super luminescent diode, halogen, plasma-arc curing (PAC), or other light source to simultaneously, sequentially, or singularly irradiate the tissues to kill or inactivate bacteria, spores, fungi, viruses, and bacteriophages and to suppress biofilm formation.

In one aspect, the blue light device irradiation is co-axial with an aiming light for guiding the laser. In another example, the blue light device comprises a separate energy source and a separate handpiece from that of the laser. Thus, the blue light device can be combined with, or completely independent from, the hardware comprising the laser.

In some embodiments, controls to perform a step of circumferentially and radially irradiating surfaces of the implant to denature or ablate bioactive bacterial products including lipopolysaccharide endotoxins. According to another example aspect, the lasing includes lasing circumferentially and radially to remove corrosion by-products of titanium oral implants, including corroded soluble debris, metal oxides, particulate debris, and metal ions resulting from metal dissolution within diseased soft tissues. In still another aspect, the lasing includes circumferentially and radially irradiating the titanium implant surfaces and threads to denature or ablate bioactive bacterial products including lipopolysaccharide endotoxins.

Ablation of the free gingival margin with the laser energy removes pathogens and pathologic proteins within the tissue of the free margin, which otherwise would not be removable. Lasing the implant surface destroys the lipopolysaccharides (LPS) of gram-negative bacteria. Additionally, this procedure provides hemostasis for better visualization, and further defines the tissue margins preceding piezoelectric instrumentation. The integrity of the mucosa is also preserved by releasing tissue tension around the implant prior to mechanical manipulation, thereby dissecting the separation between the free gingival margin and the fibrous collagen matrix, which holds the gingiva in position. Maintenance of the crest of the gingival margin is aided in that the healing of the fibrous collagen matrix will maintain the gingival crest at, or apical to, the presurgical level.

Using the quartz optical fiber oriented less than 30 degrees to the implant surface, the clinician may lase the implant surface to destroy lipopolysaccharides (LPS). Greater than 30 degrees risks the possibility that the Nd:YAG laser pulse may interact with the surface of the implant. A few pulses of Nd:YAG laser energy are not injurious to a terminal, ailing or failing implant as long as the irradiation is immediately discontinued so that heat does not accumulate within the implant. The nature of a quartz optical “bare” fiber is such that it has a 27-degree beam divergence. Therefore, even parallel to the implant surface, the Nd:YAG laser radiation can reach the surface by “side-firing” scatter.

While various embodiments of the present invention have been described above, it should be understood that they have been presented by way of example only, and not limitation. It will be apparent to those skilled in the art that various changes in the form and details can be made without departing from the spirit and scope of the invention. As such, the breadth and scope of the present invention should not be limited by the above-described exemplary embodiments, but should be defined only in accordance with the following claims and equivalents thereof.

What is claimed is:

1. A laser-based dental implant method for disinfecting and treating both the site and the implant before, during and after placement of the implant to increase predictability and success of the long-term outcome comprising the steps of:
in the oral cavity of a patient, accessing an implant site including as necessary incising soft tissue with a sterilized scalpel;
reflecting a soft tissue flap to expose alveolar bone at the implant site;
creating an osteotomy site in the bone with a sterilized rotary tool;
probing the osteotomy site at multiple locations to determine its depth;
adjusting a free length of a laser optical fiber to match the osteotomy site depth;
inserting the free length in the osteotomy site before activating an interconnected laser;
activating the laser to irradiate the osteotomy site as the free length is withdrawn from the osteotomy site;
with a free-running pulsed laser suitable for irradiating a titanium implant to achieve a hydrophilic implant surface, irradiating an implant fixture with light from the laser prior to placement in the osteotomy site;
placing the titanium implant in the osteotomy site; and,
bio-stimulating the implant site with laser light after placement of the titanium implant in the osteotomy site.

2. The method of claim 1 wherein the step of withdrawing the free length from the osteotomy site is over a time period sufficient to obtain hemostasis.

3. The method of claim 1 wherein the step of withdrawing the free length from the osteotomy site is repeated one or more times to obtain hemostasis.

4. The method of claim 1 wherein the step of withdrawing the free length from the osteotomy site is over a time period sufficient to activate activating growth factors, upregulate gene expression, and inhibit production of proinflammatory cytokines and prostaglandins.

5. The method of claim 1 wherein the step of withdrawing the free length from the osteotomy site is repeated one or more times to activate activating growth factors, upregulate gene expression, and inhibit production of proinflammatory cytokines and prostaglandins.
